(19) United States
(12) Patent Application Publication
(10) Pub. No.: US 2009/0280055 A1
(43) Pub. Date: Nov. 12, 2009

(54) Use of Fluorine-Containing Compounds for Diagnostic Purposes Using Imaging Methods

(75) Inventors: Ulrich Schrader, Dusseldorf (DE); Ulrich Flögel, Dusseldorf (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: Heinrich-Heine Universitat Dusseldorf, Dusseldorf (DE)

(21) Appl. No.: 12/089,696

(22) PCT Filed: Mar. 31, 2008
(86) PCT No.: PCT/EP08/53838

§ 371 Date: Sep. 26, 2008

(30) Foreign Application Priority Data
Mar. 29, 2007 (DE) 10 2007 015 598.2

(51) Int. Cl. A61K 51/00; A61K 49/00; A61B 5/055

(52) U.S. Cl. 424/1.11; 424/9.1; 424/9.37

(57) ABSTRACT

Use of fluorine-containing compounds for the diagnostic detection of inflammatory processes by means of an imaging method, said inflammatory processes being selected from the group consisting of inflammatory processes of the lymphatic system, such as cancers that directly affect the lymph nodes, especially Hodgkin's disease, non-Hodgkin lymphomas, tumor metastases; liver tumors, inflammatory processes in the border zone of infarctions, such as myocardial infarction and stroke, or tumors; inflammation of organs, such as myocarditis, encephalitis, meningitis (cerebral and spinal meninges); multiple sclerosis; inflammations of the gastrointestinal tract, such as Crohn's disease; inflammation of the vessels, such as arteriosclerosis, especially so-called “vulnerable plaques”; detection of abscesses and arthritis.

The present invention relates to the use of fluorine-containing compounds for the diagnostic detection of inflammatory pathological conditions using MR imaging and positron emission tomography (PET).

Inflammatory diseases are by far the most important causes of morbidity and mortality worldwide. While there are effective diagnostic and therapeutic methods for acute inflammatory diseases (predominantly caused by pathogens) in many cases, the diagnosis of chronic inflammatory diseases is mostly difficult, and the therapy thereof is limited to symptomatic measures. Non-invasive imaging methods, such as echocardiography, computer tomography and nuclear magnetic resonance spectroscopy, provide detailed anatomic information and are thus valuable tools for evaluating the function of organs. However, with none of the methods mentioned has it been possible to date to detect inflammatory processes unambiguously with high spatial resolution.

In Nature Biotechnology Vol. 23, No. 8, August 2005, p. 983-987, Arens et al. describe methods for tracking immunotherapeutic cells. By means of magnetic resonance imaging (MRI) of perfluoro-15-crown-5 ether, it is tried to detect cells in vivo that possess immunotherapeutic relevance. They used dendritic cells charged ex vivo with perfluoro-15-crown-5 ether. Subsequently, their fate after injection into mouse tissue or intravenous administration to mice was examined in vivo. WO 2005/072780 A2 and WO 03/075747 A2 relate to similar examinations. In Medica Mundi 47/1, April 2003, p. 34-39, G. M. Lanza et al. describe the use of paramagnetic nanoparticles for the targeting of cells. In this method, the paramagnetic nanoparticles are employed to produce signal extinction in the ¹H magnetic resonance image. Vehicles charged with ¹⁹F were employed as probes.

DE 694 33 723 T2 relates to a method for the in vitro delivery of biologics and to compositions that can be used in such method. Said biologics are delivered in connection with a polymeric shell.

WO-A-2005/072780 relates to the ex vivo labeling of isolated cells with perfluoro-carbons. It is disclosed that isolated cells labeled with perfluorocarbons ex vivo are suitable for studying cell migrations in the organism after injection.

DE-A-41 27 442 relates to a technology for the preparation of liposomes containing perfluorocarbons for oxygen transport, i.e., as a blood substitute.

In Biol. Pharm., Bull. 29, 9, 2006, pp. 1936-1940, Maeda, Noriyuki et al. disclose ¹⁸fluorine-deoxyglucose encapsulated in liposomes together with PET in order to examine the deposition of liposomes in tumor tissue.

Oku, Naoto et al. (Biochimica et Biophysica Acta 1280 (1996), 149-154) relates to the use of liposomes charged with ¹⁸F-labeled glucose for studying “liposomal trafficking”. The studies were performed by means of PET diagnostics.

It is the object of the present invention to provide a method which enables the visualization of specifically pathological conditions, such as inflammatory processes, by means of imaging methods, and thus the recognition of the affected regions.

The object of the invention is achieved by the use of fluorine-containing compounds for the diagnostic detection of inflammatory processes by means of an imaging method, said inflammatory processes being selected from the group consisting of pathological processes in the border zone of infarctions, such as myocardial infarction and stroke, or tumors; inflammation of organs, such as myocarditis, encephalitis, meningitis (cerebral and spinal meninges); multiple sclerosis; inflammations of the gastrointestinal tract, such as Crohn's disease; inflammation of the vessels, such as arteriosclerosis, especially so-called “vulnerable plaques”; detection of abscesses and arthritis. In addition, lymph nodes can be visualized including their pathological alterations, especially with inflammatory processes of the lymphatic system, such as cancers that directly affect the lymph nodes, especially Hodgkin's disease, non-Hodgkin lymphomas, tumor metastases, for example, from breast cancer. In particular, magnetic nuclear resonance measurement of the ¹⁹F isotope or measurement of the positron emission of the ¹⁸F isotope may be used as said imaging method. The evaluation of the corresponding measurements and conversion thereof into an image is per se known to a skilled person, as can be seen, for example, from:

Haacke M E, Brown W R, Thompson M R, Venkasetan R: Magnetic Resonance Imaging—Physical Principles and Sequence Design, Wiley, New York, 1999;

Yu J X, Kodibagkar V D, Cui W, Mason R P. ⁹F: a versatile reporter for non-invasive physiology and pharmacology using magnetic resonance; Curr Med Chem. 12: 819-48, 2005;

Wernick M N, Aarsvold J N Emission Tomography: The Fundamentals of PET and SPECT, Academic Press, London, 2004.

The method for the administration of an intravenous perfluorocarbon contrast agent for improving the echocardiographic determination of the left ventricular volume and ejection fraction as published by W. Gregory Hundley et al. in the Journal of the American College of Cardiology (JACC, Vol. 32, No. 5 (1981): 1426-32) is not covered by the subject matter of the present invention.

The invention is based on the conclusion that perfluorinated compounds, such as fluorocarbons, are taken up by monocytes/macrophages in such a way that the cells become specifically labeled, which can be utilized for diagnosis in imaging methods. Surprisingly, a diagnostic potential for the visualization of inflammatory processes and lymph nodes was found.

In one embodiment of the use according to the invention, the fluorine-containing compounds are in a vehicle. In particular, vesicles, liposomes and cyclodextrins may be used as such vehicles.

In particular, the liposomes may be unilamellar liposomes, which can be prepared and charged with fluorine-containing compounds by the skilled person in a per se known manner. It is advantageous for the liposomes to be applied in a size which is suitable for uptake by immunocompetent cells (especially macrophages), typically in a size of from 100 to 200 nm. Other liposome structures than the mentioned unilamellar ones are also possible. Liposomes and their preparation are treated in different publications and well known to the skilled person (Mozafari M R, Liposomes: an overview of manufacturing techniques. Cell Mol Biol Lett 10 (2005) 711-9; Basu S C, Basu M, Methods in Molecular Biology: Liposomes Methods and Protocols Humana Press Inc., Totowa, N.J., 2002).

In one embodiment of the use according to the invention, the fluorine-containing compounds are selected from the group consisting of inorganic or organic fluorinated, especially perfluorinated, compounds.

In particular, perfluorinated organic hydrocarbons (such as perfluorooctyl bromide, perfluorooctane, perfluorodecalin, perfluoro-15-crown-5 ether) may be used as fluorine-containing compounds. The presence of ¹⁹F and/or ¹⁸F isotopes in the fluorinated compounds allows the advantage of using devices already known and existent in the hospital, namely magnetic nuclear resonance spectroscopy with ¹⁹F isotopes and/or the use of positron emission spectroscopy of ¹⁸F isotopes.

By the use of fluorine-containing compounds according to the invention, especially when applied in a vehicle, the following pathological conditions can be detected:
1) Visualization of lymph nodes and their pathological enlargement
   a) cancers that directly affect the lymph nodes: Hodgkin's disease, non-Hodgkin lymphomas;
   b) tumor metastases, for example, from breast cancer;
   c) viral and bacterial infections, for example, syphilis, tuberculosis;
2) Inflammation reactions in the border zone of
   a) infarctions, for example, myocardial infarction, stroke;
   b) tumors;
3) inflammation of organs: myocarditis, encephalitis, meningitis (cerebral and spinal meninges);
4) multiple sclerosis;
5) inflammations of the gastrointestinal tract, for example, Crohn's disease;
6) inflammation of the vessels, for example, arteriosclerosis, especially so-called “vulnerable plaques”;
7) detection of abscesses;
8) detection of arthritis.

In the following, the invention is further illustrated by way of examples:

To date, the detection of local inflammatory processes by means of MR imaging (MRI) has been successful only when using superparamagnetic iron oxide (SPIO) particles, wherein the high affinity of such particles for the monocyte-macrophage system is utilized. Since SPIOs cause extinction of the MR signal, the data are difficult to interpret in some cases because a clear distinction from other artifacts is not possible. Therefore, it was the object of the invention to establish an MR method by which inflammatory processes could be visualized with a positive contrast for the first time. Emulsified perfluorocarbons (PFCs), which are biochemically inert and are phagocytized by the monocyte-macrophage system like SPIOs, were employed as contrast agents.

To trigger acute inflammation processes, two different lesion models (myocardial and cerebral infarction) in mice were used. The myocardial infarction was triggered by a ligature of the left (LAD) coronary artery. In a separate experimental series, the photothrombosis model was used for inducing focal ischemia. At different times after the mentioned interventions, 100-500 μl of a 10% emulsion of perfluoro-15-crown-5 ether (15C5) was injected into the caudal vein of the mouse. For the histological detection of the PFCs, a rhodamine-labeled 15C5 emulsion was used alternatively in some experiments.

Subsequently, anatomically congruent ¹H-MR and ¹⁹F-MR images were recorded with a ¹H/¹⁹F resonator (inner diameter 30 mm) at a field strength of 9.4 Tesla (Bruker DRX spectrometer; field of view 3.3 cm² (heart) and 2·2 cm² (brain), ¹H: Cine-FLASH (heart, ECG- and respiration-triggered) or RARE (brain, RARE factor 16), layer thickness 1 mm, matrix 256·256, ¹⁹F: RARE (RARE factor 64), layer thickness 2 mm, matrix 128·128.

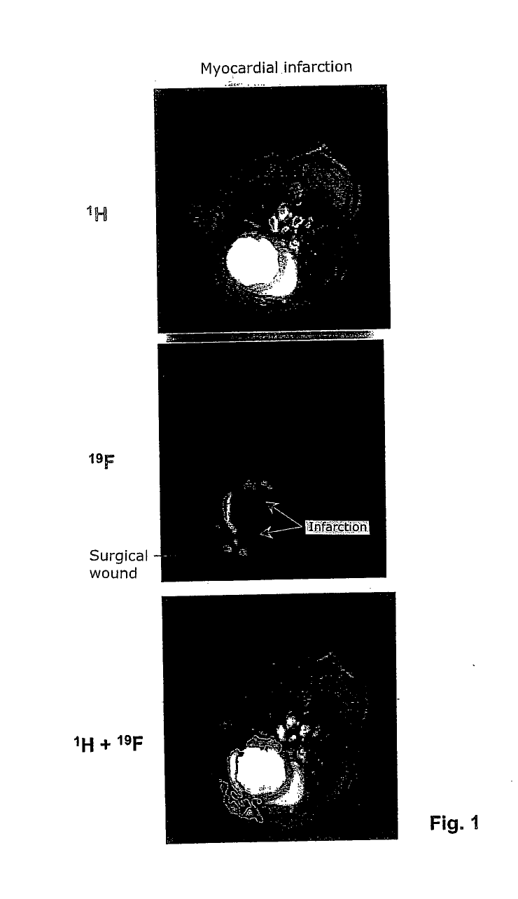

By combinedly recording ¹H and ¹⁹F images, an infiltration of PFCs into the border zones of the infarction could be detected in the heart. FIG. 1 shows anatomically congruent ¹H-MR and ¹⁹F-MR images from the thorax of a mouse four days after the triggering of a myocardial infarction (FIG. 1). The superposition clearly shows the enrichment of the fluorine signal in the infarction zone and in the surgical wound.

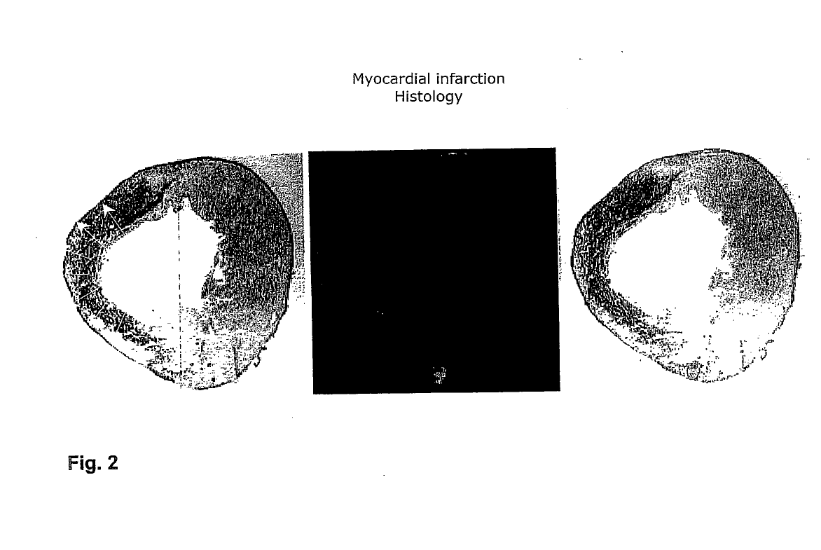

Histological bright field and rhodamine-fluorescence survey micrographs of cryosections (8 μm) of the mouse hearts recorded four days after the triggering of a myocardial infarction (FIG. 2) confirm the in vivo results. The superposition shows the localization of the rhodamine-labeled perfluorocarbons in the border zone of the infarction.

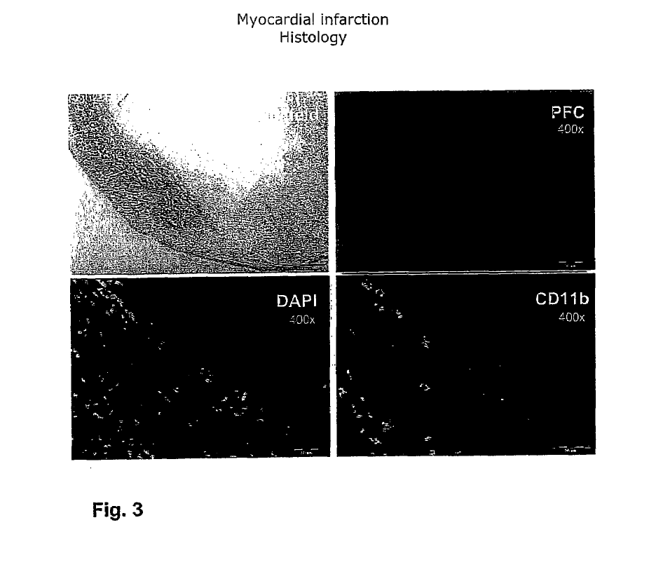

The colocalization of macrophages and PFCs could be detected by means of CD11b staining. FIG. 3 shows details of histological bright field and fluorescence micrographs of cryosections (8 μm) of a mouse heart recorded four days after the triggering of a myocardial infarction. The comparison of fluorescence clearly shows the colocalization of rhodamine labeling (PFCs) and CD11b-positive cells (monocytes/macrophages).

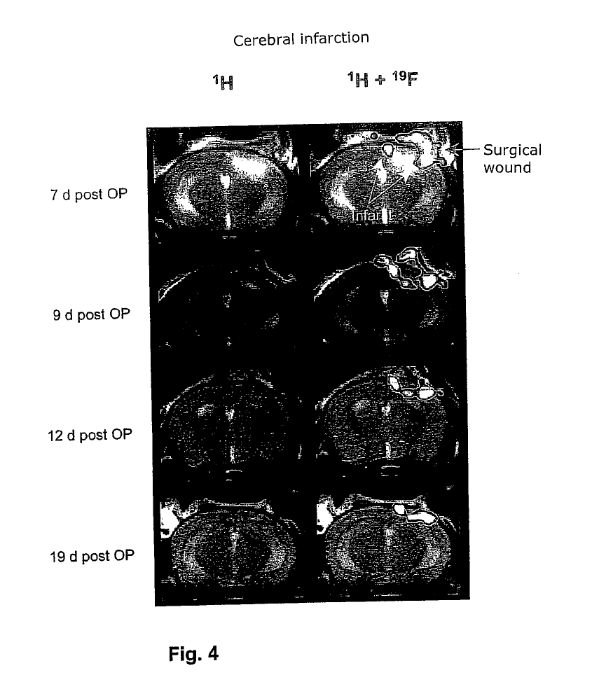

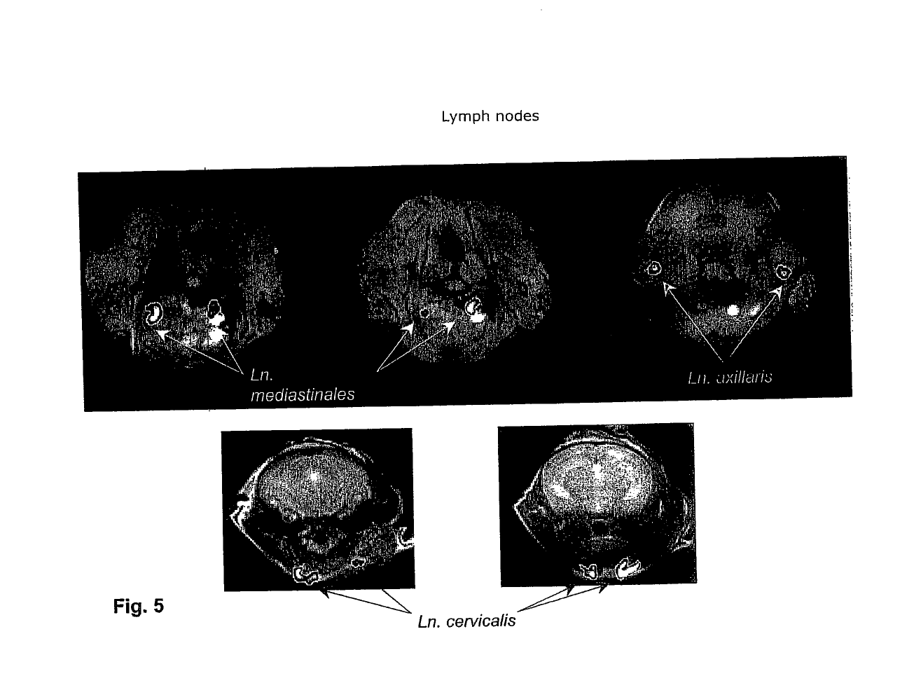

Similar results as in the heart were observed after a cerebral infarction from photothrombosis. However, the 19F signal was observed only after 6 or 7 days in the border zone of the infarction in this case (FIG. 4), which is in agreement with the delayed infiltration of the macrophages in this model as known from other studies. In this case too, additional signals could be observed in the surgical wound and in the lymph nodes (see below and FIG. 5). In addition, FIG. 4 clearly shows the shift of the PFCs together with the penumbra of the infarction area, which shrunk after some days.

Independently of induced lesions, an enrichment of the PFCs in the lymph nodes could be detected in each case. This is shown in an exemplary manner in FIG. 5 for the corresponding lymph nodes in the region of the upper chest and the head of the mouse. It is clearly seen that the perfluorocarbons become enriched in the lymph nodes present in these areas.

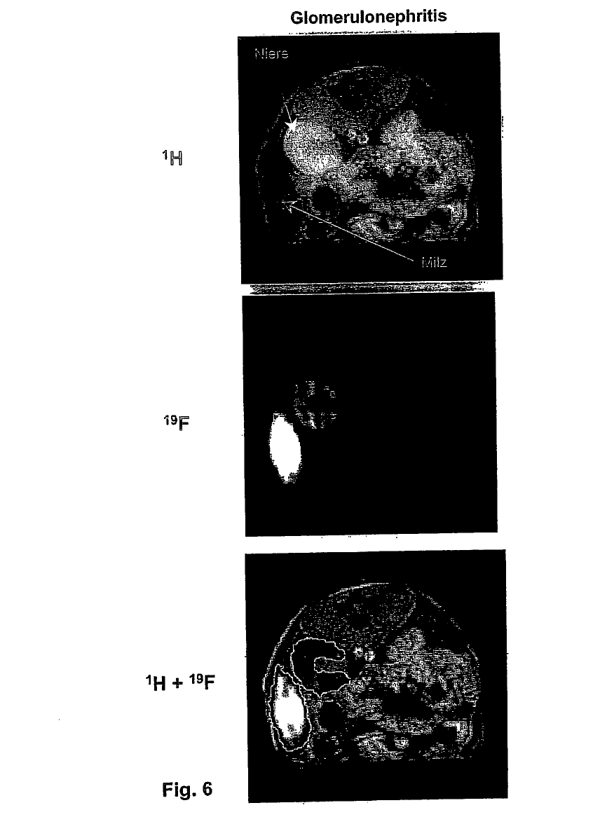

The results described were confirmed in other disease models. FIGS. 6 to 9 show that ¹⁹F-MR imaging after injection of PFC emulsions can also be employed for the visualization of inflammatory kidney diseases, for the detection of multiple sclerosis and myocarditis, and also for the visualization of inflammatory vascular diseases. In the following, the individual Figures are briefly explained:

FIG. 6 shows anatomically congruent ¹H-MR and ¹⁹F-MR images from the abdominal region of a mouse recorded 4 days after the induction of a glomerulonephritis. The superposition shows that the fluorine signal becomes enriched in the renal cortex. In addition, signals are found in the spleen (reticulo-endothelial system) as expected.

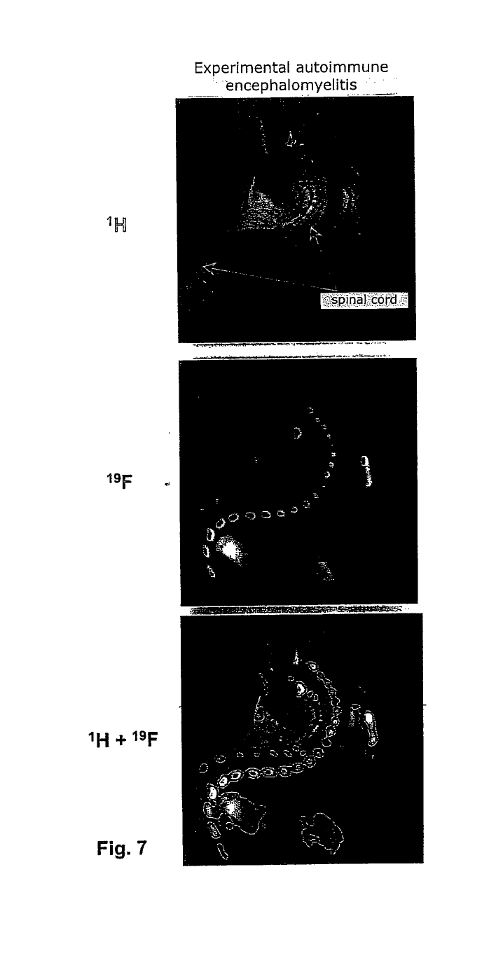

FIG. 7 shows morphologically corresponding ¹H-MR and ¹⁹F-MR images recorded 7 days after the triggering of an experimental autoimmune encephalomyelitis (animal model of multiple sclerosis), which demonstrate an accumulation of the perfluorocarbons in the spinal cord and bone marrow. In addition, signals are found in the liver (reticulo-endothelial system) as expected.

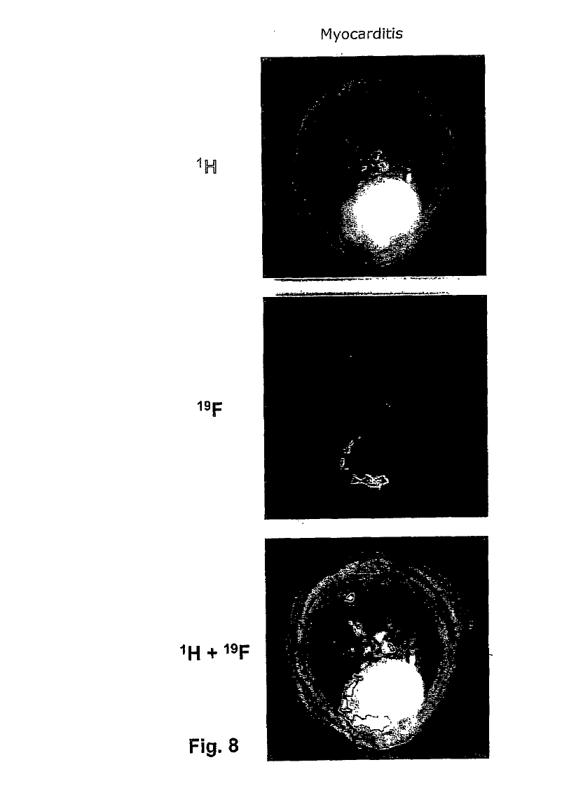

FIG. 8 shows anatomically congruent ¹H-MR and ¹⁹F-MR images which show an enrichment of the fluorine signal in the inflamed left ventricle 14 days after the triggering of a myocarditis by the injection of troponin I.

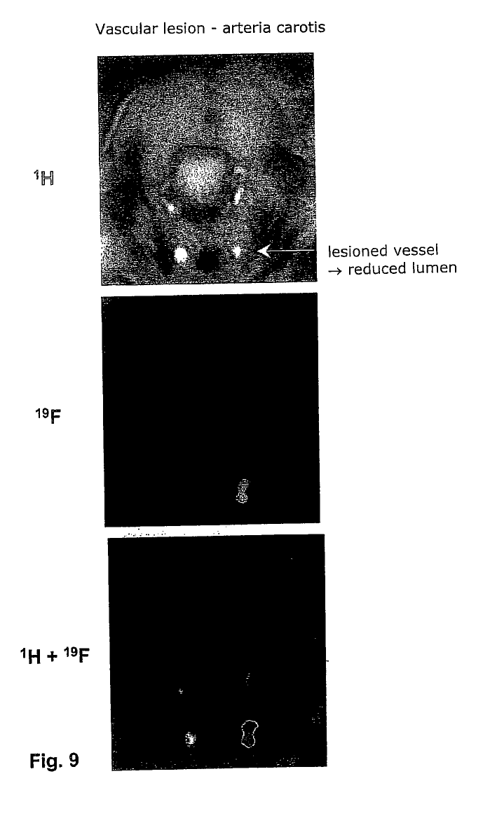

FIG. 9 shows anatomically corresponding ¹H-MR and ¹⁹F-MR images recorded 7 days after the denudation of the right arteria carotis (carotid artery, restenosis model). The perfluorocarbon signal around the reduced lumen in the damaged vessel can be clearly seen due to increased neointima formation.

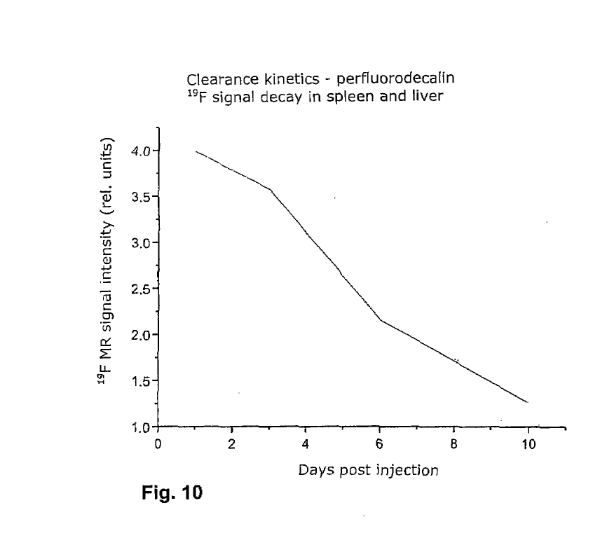

In further experiments, it was demonstrated that by varying the perfluorocarbon employed, clearance of this substance can be achieved within a few days. FIG. 10 shows the intensity of the fluorine signal in the mouse after injection of 500 μl of a 40% perfluorodecalin emulsion. After about 10 days, the perfluorocarbon has almost completely disappeared from the body.

These results show that intravenously administered emulsified PFCs accumulate in inflammatory areas and in lymph nodes after phagocytosis by the monocyte-macrophage system and can be detected by MRI with high spatial resolution. Thus, the PFCs represent a “positive” contrast agent for inflammatory processes and the lymphatic system that has a high extent of specificity due to a lack of natural ¹⁹F background. Since PFCs are known to be non-toxic, this method should also be suitable for application with humans.

1-10. (canceled)

11. A method for the detection of an inflammatory process comprising:
(a) administering a fluorine-containing compound to a subject in need thereof; and
(b) imaging the fluorine-containing compound in said subject,
wherein said inflammatory process is selected from the group consisting of:
a cancer that directly affects the lymph nodes; an inflammatory process in the border zone of an infarction or tumor; inflammation of an organ; multiple sclerosis; inflammation of the gastrointestinal tract; inflammation of the vessels; arthritis; and abscess.

12. The method of claim 11, wherein said inflammatory process is selected from the group consisting of:
Hodgkin's disease; non-Hodgkin lymphoma; myocardial infarction; stroke; myocarditis; encephalitis; meningitis; multiple sclerosis; Crohn's disease; and arteriosclerosis.

13. The method of claim 11, wherein said fluorine-containing compound is an inorganic or organic perfluorinated compound.

14. The method of claim 13, wherein said fluorine-containing compound is perfluorooctyl bromide, perfluorooctane, perfluorodecalin or perfluoro-15-crown-5 ether.

15. The method of claim 11, wherein said fluorine-containing compound comprises at least one ¹⁹F isotope or at least one ¹⁸F isotope.

16. The method of claim 15, wherein said fluorine-containing compound comprises at least one ¹⁹F isotope and at least one ¹⁸F isotope.

17. The method of claim 11, wherein said fluorine-containing compound comprises at least one ¹⁹F isotope.

18. The method of claim 11, wherein said fluorine-containing compound is localized at a site of inflammation.

19. The method of claim 11, wherein said fluorine-containing compound accumulates in immunocompetent cells by phagocytosis.

20. The method of claim 11, wherein said imaging comprises measuring said fluorine-containing compound by magnetic nuclear resonance imaging.

21. The method of claim 15, wherein said imaging comprises measuring said fluorine-containing compound by magnetic nuclear resonance imaging of the ¹⁹F isotope or by positron emission tomography of the ¹⁸F isotope.

22. The method of claim 16, wherein said imaging comprises measuring said fluorine-containing compound by magnetic nuclear resonance imaging of the ¹⁹F isotope and by positron emission tomography of the ¹⁸F isotope.

23. The method of claim 17, wherein said imaging comprises measuring said fluorine-containing compound by magnetic nuclear resonance imaging of the ¹⁹F isotope.

24. The method of claim 11, wherein said fluorine-containing compound is administered in a vehicle.

25. The method of claim 24, wherein said vehicle is selected from the group consisting of vesicles, liposomes, cyclodextrins and combinations thereof.

26. The method of claim 25, wherein said vehicle binds to nucleic acids or membranes of cells, tissues or organs in a pathological state.

27. The method of claim 26, wherein said pathological state is inflammation.

28. The method of claim 11, wherein said subject is a mammal.

29. The method of claim 28, wherein said mammal is a human.
